(19) United States
(12) Patent Application Publication
(10) Pub. No.: US 2009/0155229 A1
(43) Pub. Date: Jun. 18, 2009

(54) CHONDROCYTE-BASED IMPLANT FOR THE DELIVERY OF THERAPEUTIC AGENTS

(76) Inventor: Avner Yayon, Sitria (IL)

Correspondence Address: WINSTON & STRAWN LLP;PATENT DEPARTMENT, 1700 K STREET, N.W., WASHINGTON, DC, 20006, US

(21) Appl. No.: 11/720,849

(22) PCT Filed: Dec. 5, 2005
(86) PCT No.: PCT/IL05/01304

§ 371 Date: Mar. 5, 2009

Related U.S. Application Data
(60) Provisional application No. 60/633,163, filed on Dec. 6, 2004.

(51) Int. Cl. A61K 45/00; A61P 35/00

(52) U.S. Cl. 424/93.21

(57) ABSTRACT

The present invention relates in general to chondrocyte based explants and implants of genetically engineered chondrocytes and in particular, to the delivery of peptides, proteins and RNAi molecules to a mammalian subject using a genetically modified chondrocyte-based mass. In one embodiment the genetically modified chondrocyte-based mass is provided as a chondrocyte pellet.

### FIELD OF THE INVENTION

The present invention relates in general to genetically engineered chondrocytes and in particular, to the delivery of bioactive molecules including peptides, proteins and RNAi molecules to a mammalian subject using a genetically modified chondrocyte-based implant. In one embodiment the genetically modified chondrocyte-based implant is provided as a chondrocyte pellet.

### BACKGROUND OF THE INVENTION

### Gene Delivery

Therapeutic agents may be delivered to a subject by various methods, including orally, transdermally, by inhalation, by injection and by depot. The method of delivery is determined by the required administration frequency, the nature of the disease and the target tissue. One widely investigated approach to drug delivery is the use of genetically modified cells for the delivery of therapeutic gene products to a subject. A variety of cell types and vectors has been tested for this purpose. For example, Pereboeva et al. (2003) teach the use of mesenchymal progenitor cells as cellular vehicles for the delivery of therapeutic genes or viruses to tumor sites.

Furthermore, gene delivery may be performed directly. U.S. Pat. Nos. 5,763,416 and 5,942,496 relate to methods, compositions and devices for use in transferring nucleic acids into bone cells in situ useful to promote bone growth, repair and regeneration in vivo.

Currently, the clinical application of genetically modified cells or tissue is limited for several reasons, including the short-lived nature of the gene expression. The DNA introduced into cells must remain functional and the cells must be robust, stable, non-immunogenic and contained.

The minimal prerequisites for favorable therapy are 1) an appropriate level of gene expression for a prescribed time, and 2) safe delivery and expression of the gene. Three major approaches to gene delivery include viral vectors, nonviral vectors, and physical gene transfer. Viral vectors are currently the most effective means for efficient gene transfer. Viruses can be manipulated to remove their disease-causing genes and insert therapeutic genes. Cells are infected with the viral vector, which unloads its genetic material containing the therapeutic gene into the cell. The cell manufactures a functional peptide or protein product from the therapeutic gene and secretes a functional therapeutic peptide to the milieu.

Different types of mammalian viruses are useful as vectors including retroviruses, adenovirus (AV), adeno-associated viruses (AAV) and Herpes simplex viruses (HSV).

### Tissue Grafts and Explants

The use of fetal intact tissue or tissue explants for tissue repair to a subject is taught in U.S. Pat. No. 5,976,524, WO 2004/016276, US 2003/0198628, US 2004/0082064, among others. These disclosures do not relate to genetically modified explants or grafts. International (PCT) patent application publications WO 03/035851 and WO 03/049626 teach a genetically modified micro-organ explant useful for transplantation and the delivery of gene products to a recipient. In certain embodiments, the micro-organ culture is isolated from lymphoid organs, digestive tract organs, skin, and others. The applications specifically disclose that the microarchitecture of the organ is maintained in culture.

The above patent applications neither teach nor suggest a cartilage explant or chondrocyte based implant for the delivery of gene products.

### Chondrocytes

Chondrocytes are specialized cells that are capable of producing the components of cartilage tissue, including the extracellular matrix. The biochemical composition of cartilage differs according to type but in general comprises collagen, predominantly type II collagen along with other minor types, e.g., types V, VI, IX and XI, proteoglycans, other proteins and water. Several types of cartilage are recognized in the art, including, for example, hyaline cartilage, articular cartilage, costal cartilage, fibrous cartilage (fibrocartilage), meniscal cartilage, elastic cartilage, auricular cartilage, and yellow cartilage.

Methods for the delivery of foreign DNA into chondrocytes are known in the art. U.S. Pat. No. 6,803,234 teaches a method for the delivery of a nucleic acid into a primary chondrocyte comprising providing a recombinant adenovirus having a tropism for a human chondrocyte. The preferred recombinant adenovirus vector is based on adenovirus serotype 5 with modified fiber genes. The method is further directed to a pharmaceutical composition for use in the treatment of cartilage diseases. The patent neither teaches nor suggests the use of a chondrocyte based culture system as a production depot for the delivery of therapeutic proteins to heterologous organs.

US patent application 20050124038 provides methods for transfecting and/or transducing neocartilage or juvenile cartilage with a recombinant vector, preferably adenovirus fiber type 51.

U.S. Pat. No. 6,315,992 relates to a method of generating hyaline cartilage in a mammal comprising injecting to a joint space a population of fibroblast cells that have been transduced with a recombinant vector comprising a DNA sequence encoding transforming growth factor β1 (TGF-β1) operatively linked to a promoter.

Arai et al (2004) teach a method for the adenoviral delivery of genes to primary chondrocytes, followed by three-dimensional pellet culture useful to assess the role of certain genes on cartilage matrix synthesis and degradation. Arai et al. (2000) disclose an efficient method of gene transduction to human chondrocytes using an adeno-associated virus vector.

Ikeda et al (2000) teach the transfection of chondrocytes using an adenovirus vector, for the delivery of gene products to a joint and the treatment of cartilage defects.

Madry et al., (2003) teaches direct gene transfer into normal and osteoarthritic articular cartilage for gene therapy of articular joint disorders. Recombinant adeno-associated vectors (rAAV) are capable of effecting gene transfer when applied in vivo to femoral chondral defects and osteochondral defects in a rat knee model. The above references neither teach nor suggest a chondrocyte-based implant useful for the delivery of therapeutic agents to heterologous sites in a subject.

There remains a yet unmet need for a safe and efficient cell-based system useful for delivering of gene products to a recipient. The cell-based system will ideally comprise a non-immunogenic universal cell source that is readily isolated and manipulated.

### SUMMARY OF THE INVENTION

The present invention provides a chondrocyte-based explant or an implant comprising genetically modified chondrocytes useful for the delivery of a bioactive molecule to a recipient. According to one aspect the chondrocytes are genetically modified to express an exogenous therapeutic agent. According to another aspect the genetically modified chondrocytes are cultured to form a condensed chondrocyte mass, which produces the therapeutic agent. According to another aspect the present invention provides methods of transplanting to a subject in need of a therapeutic agent a genetically modified chondrocyte explant or cells derived therefrom or a mass of such cells.

According to one aspect the present invention provides a cell mass comprising a plurality of genetically modified chondrocytes, wherein the genetically modified chondrocytes express a therapeutic agent. In one embodiment the cell mass is selected from a mass formed from dispersed genetically modified chondrocytes, a genetically modified chondrocyte based explant, and a mass formed from cells derived from a genetically modified chondrocyte based explant. In another embodiment the cell mass further comprises non-chondrocytic cells while substantially retaining its cartilage characteristics. The cell mass is formed from a mixture of genetically modified chondrocytes and other types of genetically modified cells. As non-limiting specific embodiments such other cells may be fibroblasts, pancreatic β islet cells or dopamine secreting cells.

In certain embodiments the cell mass is formed from dispersed genetically modified chondrocytes.

In one embodiment the chondrocytes are derived from articular cartilage. In another embodiment the chondrocytes are derived from stem cells, embryonic stem cells, chondroprogenitor cells or mesenchymal progenitor cells (MPC). In another embodiment the chondrocytes are selected from primary cells or a cell line. In one specific embodiment the condensed cell mass is a chondrocyte based explant or a chondrocyte pellet.

In another embodiment the chondrocytes are isolated from a source selected from an autologous source, an allogeneic source and a xenogeneic source. In certain embodiments the chondrocytes are isolated from an autologous source.

In yet another embodiment the chondrocytes are genetically modified using a gene delivery vehicle selected from a viral vector and a non-viral agent. In certain embodiments the gene delivery vehicle is a viral vector selected from adenovirus, adeno-associated virus and a retrovirus.

In certain embodiments the cell mass provides delivery of the therapeutic agent useful for treating a disease or disorder in a subject. In some embodiments the cell mass transplanted at a heterologous site in a subject for delivery of a therapeutic agent. A heterologous site refers to a site of a subject other than a site normally populated with chondrocytes.

In yet another embodiment the therapeutic agent is selected to induce or stimulate a cellular function selected from cell division, cell growth, cell proliferation and cell differentiation. In another embodiment the therapeutic agent is selected to inhibit a cellular function selected from cell division, cell growth, cell proliferation and cell differentiation.

In certain embodiments the therapeutic agent is selected from a peptide, a protein and a RNAi. In specific embodiments the therapeutic agent is a protein.

In certain embodiments the therapeutic peptide or protein is selected from a growth factor, a growth factor receptors, a hormone, an antibody, a ribozyme, a protein hormone, a peptide hormone, a cytokine, a cytokine receptor, a pituitary hormone, a clotting factor, an anti-clotting factor, a plasminogen activator, an enzyme, an enzyme inhibitor, an extracellular matrix protein, an immunotoxin, a surface membrane protein, a T-cell receptor transport protein, a regulatory proteins and fragments thereof. In specific embodiments the therapeutic protein is an antibody.

In one embodiment the disease or disorder is an acquired or genetic deficiency including diabetes, Gaucher's disease, Fabry disease and tumors. Certain tumors may arise as the result of a genetic deficiency, including tumors having cells that have lost a tumor suppressor gene such as p53, BRCA1 and Rb.

In another embodiment the disease or disorder is an acquired or genetic gain of function disease or disorder including achondroplasia and tumors.

In yet another embodiment the disease or disorder is selected from a cartilage or bone disease or disorder, a brain disorder, a cardiovascular disorder, a pulmonary disorder, a muscular disorder, a lymphatic system disorder.

In certain embodiments the condensed cell mass provides a therapeutic agent ex vivo. In specific embodiments the condensed cell mass is transplanted to a subject in need of a therapeutic agent.

In certain embodiments the subject is a mammal. In specific embodiments the subject is a human.

According to one aspect the present invention provides a cell mass comprising a plurality of genetically modified chondrocytes, wherein the genetically modified chondrocytes express a therapeutic agent. In one embodiment the cell mass is selected from a mass formed from dispersed genetically modified chondrocytes, a genetically modified chondrocyte based explant, and a mass formed from cells derived from a genetically modified chondrocyte based explant.

Therefore, according to another aspect the present invention provides methods of transplanting to a subject in need of a therapeutic agent a cell mass comprising a plurality of genetically modified chondrocytes, wherein the genetically modified chondrocytes express a therapeutic agent.

According to one embodiment the present invention provides a method for transplanting to a subject in need of a therapeutic agent a cell mass comprising a plurality of genetically modified chondrocytes, the method comprising the steps of:
a. isolating a cartilage explant;
b. transducing cells of the explant to form genetically modified chondrocytes;
c. transplanting the genetically modified chondrocytes into a subject,

wherein the genetically modified chondrocytes express the therapeutic agent.

The present invention further provides a method of transplanting to a subject in need of a therapeutic agent wherein the cell mass is selected from a mass formed from dispersed genetically modified chondrocytes, a genetically modified chondrocyte based explant, and a mass formed from cells derived from a genetically modified chondrocyte based explant.

In other embodiments the present invention provides a method for transplanting to a subject in need of a therapeutic agent an implant comprising genetically modified chondrocytes, the method comprising the steps of:
a. providing genetically modified chondrocytes;
b. inducing formation of a condensed cell mass; and
c. transplanting the condensed cell mass into a subject.

According to one embodiment the genetically modified cells are derived from a genetically modified cartilage explant. In other embodiments the genetically modified cells are derived from dispersed chondrocytes.

These and further features of the present invention will be better understood in conjunction with the drawings, detailed description, examples and claims that follow.

### BRIEF DESCRIPTION OF THE FIGURES

These and other features, aspects and advantages of the present invention will become better understood with reference to the following description, appended claims and accompanying figures where

FIG. 1 shows the cross section of a chondrocyte pellet culture stained with anti-collagen II antibody.

FIG. 2 shows the cross section of a pellet culture stained with toluidine blue.

FIG. 3 shows the cross section of a mixed pellet culture stained with alcian blue 3 weeks post preparation. A. a cell pellet consisting of 100% fibroblasts. B. a cell pellet consisting of 50% chondrocytes and 50% fibroblasts.

FIG. 4 shows the transfection of a chondrocyte cell line with an EGFP vector.

### DETAILED DESCRIPTION OF INVENTION

The present invention is directed to a genetically modified chondrocyte-based explant or an implant comprising genetically modified chondrocytes useful for the delivery of gene expression products to a subject. The explant and implant act as depots for the delivery of bioactive molecules including proteins, peptides and RNAi molecules. Therapeutic peptides and proteins include in a non-limiting manner growth factors and antibodies, useful for the treatment of a variety of diseases and disorders.

In one embodiment the chondrocytes are transduced with a nucleic acid encoding an exogenous therapeutic agent and cultured to form a condensed chondrocyte mass that can be transplanted to a subject in need of said therapeutic agent. In other embodiments a chondrocyte based explant is transduced with a nucleic acid encoding an exogenous therapeutic agent and the genetically modified explant may be transplanted to a subject in need of said therapeutic agent. For convenience certain terms employed in the specification, examples and claims are described herein.

The term “explant” as used herein refers to a group of cells isolated from an organ and kept in vitro so as to preserve its inherent architecture. Tissue and cell culture preparations of explants, isolated cells and progenitor cell populations can take on a variety of formats. For instance, cells can proliferate in a cell culture plate or flask, or in a “suspension culture” in which cells are suspended in a suitable medium. Likewise, a “continuous flow culture” refers to the cultivation of cells or explants in a continuous flow of fresh medium to maintain cell growth and or proliferation.

A “vector” is a replicon, such as a plasmid, phage or virus, to which another nucleic acid sequence may be joined in order to cause the expression of the joined nucleic acid. The nucleic acid sequence may encode a protein or peptide or alternatively may provide an RNAi molecule including dsRNA and siRNA.

A “host cell” is a cell used to propagate a vector and its insert. Transduction of the cell can be accomplished by methods well known to those skilled in the art, for example, using a viral vector or non-viral techniques including liposomes or direct insertion.

A DNA “coding sequence” is a DNA sequence, which is transcribed and translated into a peptide or polypeptide in vivo when placed under the control of appropriate regulatory sequences. A coding sequence can include, but is not limited to, prokaryotic sequences, cDNA from eukaryotic mRNA, genomic DNA sequences from eukaryotic (e.g., mammalian) DNA, viral DNA, and even synthetic DNA sequences.

A “promoter sequence” is a DNA regulatory region capable of binding RNA polymerase or an auxiliary protein and initiating transcription of a coding sequence. In general, the promoter sequence is in close proximity to the 5′ terminus by the translation start codon (ATG) of a coding sequence and extends upstream (5′ direction) to include the minimum number of bases or elements necessary to facilitate transcription at levels detectable above background. The promoter sequence typically comprises a transcription initiation site, as well as protein binding domains responsible for the binding of RNA polymerase. Other regulatory elements including “TATA” boxes and “CAAT” boxes may be present.

A coding sequence is “operably linked to” or “under the control of” a promoter or control sequences in a cell when RNA polymerase will interact with the promoter sequence directly or indirectly and result in transcription of the coding sequence.

Chondrocytes and Chondroprogenitor cells

Cartilage is categorized into three general subgroups, hyaline, elastic, and fibrocartilage, based primarily on morphologic criteria and secondarily on collagen (Types I and II) and elastin content.

Certain properties of chondrocytes and chondroprogenitor cells render the chondrocyte-based cell implant advantageous over other cell based gene delivery systems. The advantages of chondrocytes and chondroprogenitor cells include:
a) Easily isolated cells and tissue. Different types of cartilage may be used as a source of chondrocytes including articular and hyaline cartilage;
b) Readily available tissue. Chondrocytes may be isolated from a variety of sources including allogeneic, autologous and xenogeneic sources;
c) Non-immunogenic tissue. Cartilage and chondrocytes embedded within cartilaginous matrix are immune-privileged, thus providing a universal cell source;
d) Safe tissue. Chondrocytes do not undergo transformation spontaneously and proliferative disorders of cartilage are extremely rare;
e) Naturally adhesive. Chondrocytes produce adhesion molecules and extracellular matrix that facilitates cellular aggregation into a stable mass in culture.

### Genetically Modified Cells

The present invention is not limited by the foreign genes or coding sequences (prokaryotic and eukaryotic) that are inserted into the cells. The chondrocytes can be modified to express a recombinant protein or other therapeutic agent, which may or may not be normally expressed by chondrocytes.

For example, the chondrocytes may be modified to produce gene products normally produced by the pancreas, for example insulin, amylase, protease, lipase, trypsinogen, chymotrypsinogen, carboxypeptidase, ribonuclease, deoxyribonuclease, triacylglycerol lipase, phospholipase A2 and elastase. Likewise, the chondrocytes may be modified to produce gene products normally produced by the liver, including blood clotting factors, such as blood clotting Factor VIII and Factor IX and UDP glucuronyl transferase. Gene products normally produced by the thymus include serum thymic factor, thymic humoral factor, thymopoietin and thymosinal. A gene product normally produced by the kidney includes erythropoietin.

Specific examples of proteins that can be expressed in this system include but are not limited to growth factors and polypeptide hormones and other proteins that can stimulate various cellular processes concerning cell division, cell growth, cell proliferation, and cell differentiation and the like.

The following non-limiting examples illustrate various types of growth factors and growth factor receptors, protein and peptide hormones and receptors, cytokines and cytokine receptors, agonists or antagonist of a growth factor or hormone receptor that can be used: proinsulin, insulin like growth factor-1 and insulin like growth factor-2, insulin A-chain; insulin B-chain, platelet derived growth factor, epidermal growth factor, fibroblast growth factor, nerve growth factor or other neurotrophic factors such as brain-derived neurotrophic factor (BDNF), neurotrophin-3, -4, -5, or -6 (NT-3, NT-4, NT-5, or NT-6), vascular endothelial growth factor, a colony stimulating factor e.g., M-CSF, GM-CSF, and G-CSF, transforming growth factor and TGF-β related proteins such as inhibin, activin or Mullerian-inhibiting substance, tumor necrosis factor, bone morphogenic proteins (BMPs), angiotensin, calcitonin, glucagons, leptin, parathyroid hormone, growth hormone, growth hormone releasing factor, mouse gonadotropin-associated peptide, gonadotropin, relaxin A-chain, relaxin B-chain, prorelaxin, a natriuretic peptide such as atrial natriuretic factor and brain natriuretic peptide-32, a hematopoeitic cytokine such as erythropoietin, granulocyte-colony stimulating factor (G-CSF) or leukemia inhibitory factor (LIF), interleukins (ILs), e.g., IL-1 to IL-17 or an interferon such as interferon-alpha, -beta, and -gamma or their corresponding receptors, or other cytokines such as RANTES, MIP-1 alpha or MIP-1 beta.

Additional heterologous proteins include a pituitary hormone such as bombesin, corticotropin releasing factor (CRF), follicle stimulating hormone, oxytocin, somatotropin or vasopressin; a clotting factor such as factor VIIIC, factor IX, tissue factor, and von-Willebrand factor; an anti-clotting factor such as Protein C; a plasminogen activator such as urokinase or tissue-type plasminogen activator, including human tissue-type plasminogen activator (t-PA) or thrombin; an enzyme such as caspases, calpains, cathepsins, DNase, enkephalinase, matrix metalloproteinases (MMP) superoxide dismutase, alpha-galactosidase A and protein kinases or an enzyme inhibitor exemplified by plasminogen activated inhibitor-1 or cathepsin inhibitor; an extracellular matrix protein such as a collagen or a fibronectin; a serum albumin such as human serum albumin; a microbial protein, such as beta-lactamase; a CD protein such as CD-3, CD-4, CD-8, and CD-19; immunotoxins; a surface membrane proteins; a T-cell receptor; a viral antigen such as, for example, a portion of the AIDS envelope; transport proteins; regulatory proteins; antibodies; and fragments of any of the above-listed polypeptides.

Currently most preferred examples of proteins expressed using the high yield expression system includes, but is not limited to the FGF family of proteins and FGF receptor antibodies.

### Gene Delivery Vehicle

Different types of viruses are useful as vectors including:
a) Retroviruses: a class of RNA viruses that can create double-stranded DNA copies of their RNA genomes. The DNA can integrate into the host cell chromosomes.
b) Adenoviruses (AV): a class of viruses with linear double-stranded DNA that do not integrate into host chromosomal DNA and remain an episome in cells.
c) Adeno-associated viruses (AAV): a class of small parvoviruses, which can insert their single-stranded DNA at a specific site on human chromosome 19.
d) Herpes simplex viruses: a class of double-stranded DNA viruses that infect neurons.
e) Vaccinia viruses: a class of double-stranded DNA viruses, which remain in the cytoplasm of infected cells. Vaccinia virus infects nearly all mammalian cell types but may induce a strong cytotoxic T-cell response in tissue.

There are many types of retroviruses including murine leukemia virus (MLV), human immunodeficiency virus (HIV), equine infectious anemia virus (EIAV), mouse mammary tumour virus (MMTV), Rous sarcoma virus (RSV), Fujinarni sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al 1997).

The retroviruses contain three major coding domains, gag, pol, env, which code for essential virion proteins. Nevertheless, retroviruses may be broadly divided into two categories: namely, “simple” and “complex”. These categories are distinguishable by the organization of their genomes.

The present invention also contemplates mutant viruses, such as those disclosed in US patent application 20040234549. U.S. Pat. No. 6,140,087 discloses a series of adenovirus-based vectors having deletions in the E1 and/or E3 regions, and also insertions of pBR322 sequences, which can be used to deliver nucleic acid inserts into host cells, tissues or organisms that then can express the insert.

The nucleic acid encoding a therapeutic agent carried by the recombinant virus can be operatively linked to any heterologous or homologous promoter that is commonly used in the art to drive the transcription and/or translation of a heterologous nucleic acid. In certain embodiments the promoter is either a CMV, CMV-IE, TK, SV40, T7, Sp6, EM7, bla, Actin, collagen, metallothionein (MT), EF-1 alpha, TET, an ecdysteroid responsive promoter, MMTV, HSV, HSV-IE 175, MuLV, RSV, EF-1, or a baculovirus promoter. The promoter is used by the heterologous polynucleotide to direct and regulate its transcription and/or translation.

The present invention contemplates gene delivery using nonviral methods. One nonviral approach involves the creation of an artificial lipid sphere with an aqueous core. This liposome, which carries the therapeutic DNA, is capable of passing the DNA through the target cell's membrane.

Therapeutic DNA can also get inside target cells by chemically linking the DNA to a molecule that will bind to special cell receptors. Once bound to these receptors, the therapeutic DNA constructs are engulfed by the cell membrane and passed into the interior of the target cell. This delivery system tends to be less effective than other options.

Dinser et al., (2001) compared long-term transgene expression in chondrocytes after viral and nonviral gene transfer. Adenovirus was compared to plasmid transfection, and both were shown to be useful. Madry and Trippel (2000) teach lipid-mediated gene transfer for transfection of articular chondrocytes.

### RNA Inhibiting Molecules

Selection of RNAi sequences for the effective inhibition of RNA is well known to one skilled in the art. For example, guidelines for the selection of highly effective siRNA sequences for mammalian RNA interference are described in Ui-Tei et al. (2004).

The ability of a RNA interference molecule containing a given target sequence to cause RNAi-mediated degradation of the target mRNA can be evaluated using standard techniques for measuring the levels of RNA or protein in cells. For example, siRNA of the invention can be delivered to cultured cells, and the levels of target mRNA can be measured by Northern blot or dot blotting techniques, or by quantitative RT-PCR. Alternatively, the levels of a therapeutic protein produced by the cultured cells can be measured by ELISA or Western blot.

Degradation of the target mRNA by an RNAi molecule reduces the production of a functional gene product. Thus, the invention provides a method of inhibiting expression of certain proteins in a subject, comprising administering an effective amount of an RNAi molecule of the invention to the subject, such that the target mRNA is degraded.

### Method of Preparing Implant

The present invention is not limited by method of preparing the implant. In one embodiment the implant is a genetically modified cartilage explant. The explant maybe genetically modified in situ or ex vivo and may be isolated from a subject by methods known in the art including biopsy.

In other embodiments the implant derives from cells isolated from a genetically modified explant. The cells may be isolated for example by enzymatic digestion of an explant. In other embodiments the implant comprises chondrocytes isolated from an explant, and the chondrocytes expanded and transduced in vitro.

The cell mass' potential to deliver recombinant proteins may be increased by mixing into the chondrocytes other cell types that may be more efficiently transduced by the virus, or may have a unique cellular machinery suitable for the expression and secretion of certain proteins. The mixed product would therefore contain chondrocytes with the potential of forming cartilage cell pellets together with other cells that can be efficiently transduced. The mixed cell mass substantially retains its cartilage characteristics as can be measured by staining tissue sections of the cell mass using multiple cartilage markers well known in the art, e.g. collagen 2, Alcian Blue and Safranin-O.

Non limiting examples of such cells are fibroblasts, endothelial cells, β islet cells, or liver cells.

### Applications

The therapeutic products produced using the method of the present invention are intended to be delivered in vivo but can be used to produce a therapeutic agent in vitro. The chondrocyte mass may be implanted at a variety of sites within a subject. In one embodiment the chondrocyte mass is implanted near a fracture in a bone for delivery of growth factors useful for treatment of a bone fracture. In another embodiment the chondrocyte mass is implanted in a subject for delivery of a hormone, including insulin or erythropoietin.

A transplanted cell mass comprising genetically modified chondrocytes may undergo vascularization by the host's cells. Without wishing to be bound by theory, vascularization will assist in the delivery of the therapeutic agent to the target tissue or organ.

### EXAMPLES

Although certain preferred embodiments of the present invention have been described, the spirit and scope of the invention is by no means restricted to what is described above.

### Example 1

### Articular Chondrocyte Culture

Chondrocytes were isolated from pig or human biopsies and cultured according to the procedure presented below.

### Reagents:

Dulbecco's MEM (DMEM) (Gibco BRL)

MEM Non-Essential Amino Acids (Gibco BRL)

Sodium Pyruvate (Gibco BRL)

Fetal Bovine Serum (FBS) (Gibco BRL)

Streptomycin, Penicillin, Nystatin Solution (Biological Indus.)

Trypsin-EDTA (Gibco BRL) or Versene-Trypsin (Bio LAB Ltd.)

Collagenase Type 2 (Worthington Biochem. Corp.) A stock solution of 1700 units/ml

Collagenase in DMEM was prepared and filtered (0.2 μm).

### Preparation of FBS-DMEM Medium:

FBS (50 ml), 5 ml of antibiotic solution, 5 ml Sodium Pyruvate, 5 ml MEM non-essential amino acids were added to a 500 ml bottle of DMEM. Where specified, FGF growth factors were added to a final concentration of 10 ng/ml.

Isolation of Cells from Cartilage Biopsy:

In certain embodiments, chondrocytes are isolated from a cartilage explant, prior to transduction. A piece of cartilage tissue was minced into 1 to 2 mm pieces with a sterile scalpel. The collagenase solution was diluted 1:4 in FBS-DMEM, added to the tissue sample and left to incubate on a rotator at 37° C., overnight (ON). The cells were centrifuged (1200 rpm 5-10 min). The medium was aspirated, the cells washed in 5 ml medium and recentrifuged. The cells were resuspended in culture medium and seeded in 25 cm² or 75 cm² flasks at a concentration of approximately 1×10⁶ cells per flask. The cells were incubated in a 5% CO₂ incubator at 37° C. The cell medium was replaced every 2-3 days.

### Procedure for Passaging Cells (Trypsinization):

When the cell culture reached the desired confluency the medium was removed and the cells trypsinized according to standard procedure. The cells were split to 2-3 new flasks and 20 ml fresh pre-warmed medium was added. The expansion of cells and trypsinization was performed as necessary.

Furthermore, the cell population grown on the above matrices expresses several of the chondrocyte differentiation markers. One of several phenotypes expressed during chondrocyte differentiation is glycosaminoglycan (GAG) production. The production of GAGs is identified in histological staining using Alcian blue or toluidine blue and quantitated using the DMB (3,3′-dimethoxybenzidine dihydrochloride) dye method.

### Example 2

### Cell Proliferation/Differentiation Assay

Articular chondrocytes that have been isolated by enzymatic digestion and maintained in monolayer culture undergo dedifferentiation over time and shift to a fibroblast-like phenotype. This is reflected in part by their morphology and loss of expression of collagen II. The cells are able to undergo proliferation and differentiation into articular chondrocytes under certain growth conditions.

Proliferation of the cartilage cells was quantitated by one of two methods, CyQUANT® (Molecular. Probes) or XTT reagent (Biological Industries, Co.). Human or porcine articular chondrocytes (10⁴-10⁵ cells/100 ul) were grown in microwell plates for several days in DMEM with and without growth factors, and the cells processed according to manufacturers instructions. The plates were read in an ELISA reader at A490 nm.

Articular chondrocytes were isolated from cartilage tissue fragments. Dispersed cells were grown using culture media supplemented with Fetal Calf Serum (FCS) with FGF growth factors. Medium was exchanged every 2-3 days. Proliferation of cells was determined using CyQUANT™ Cell Proliferation Assay Kit (Molecular Probes).

### Example 3

### Chondrocyte Pellet Culture

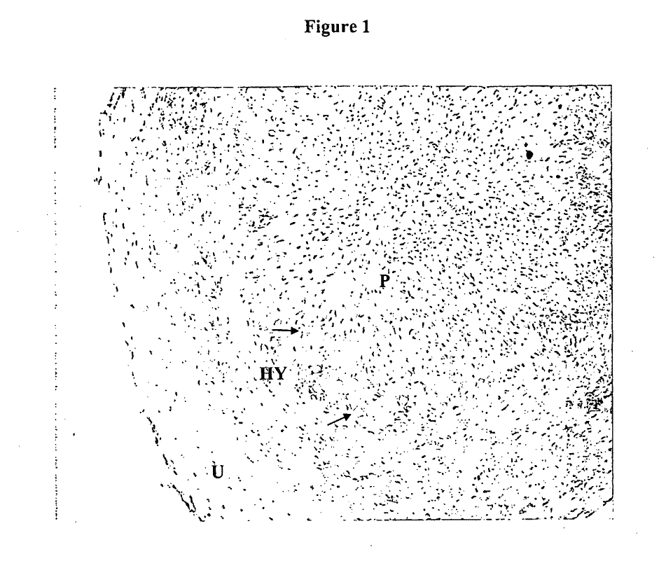

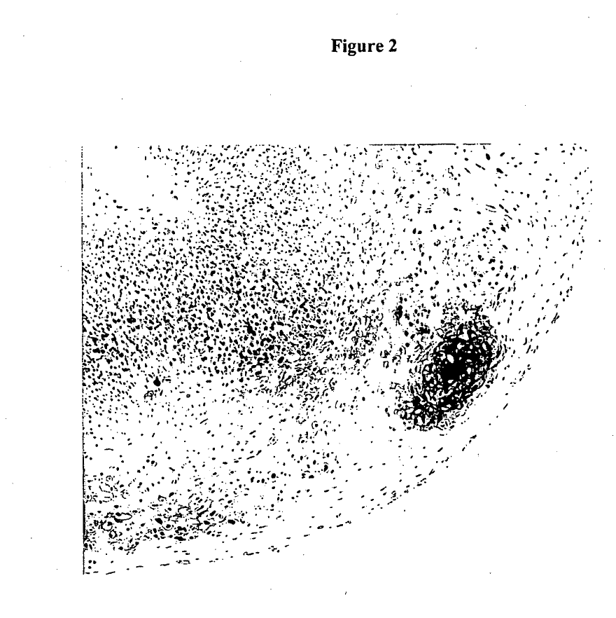

Typically, dispersed chondrocytes that are cultured in vitro, proliferate and exhibit reduced collagen II expression. Cell differentiation and morphogenesis was studied in pellet cultures and analyzed by using cell-type-specific markers. 2.5×10⁵ porcine articular chondrocytes that had been expanded in culture were pelleted in 0.5 ml differentiation medium (DMEM-high glucose containing the following: 1 μM dexamethasone, 1 mM Sodium pyruvate, 50-100 ug/ml ascorbic acid, 0.35 mM proline, 10 ng/ml IGF-1, 10 ng/ml TGFβ, Insulin-Transferrin-Selenium solution (6.25 μg/ml each)) and incubated in differentiation medium in 15 ml polypropylene centrifuge tubes with caps loosened. Medium was replaced every 2-3 days. The pellets were sectioned using standard methods known in the art and stained with toluidine blue to label the sulfated proteoglycans and immunohistochemically stained with anti-collagen II antibodies. FIGS. 1 and 2 show histological sections of chondrocyte pallets. FIG. 1 shows a section stained with an anti-Collagen II antibody. The letter “U” refers to the small layer of undifferentiated cells surrounding the pellet. “HY” refers to the thick layer of mature hypertrophic chondrocytes. Note the large lacunae and the darker color indicating collagen II staining. “P” refers to the core of proliferating chondrocytes.

FIG. 2 shows a cross section of a chondrocyte pellet stained with toluidine blue.

### Example 4

### Cell Pellet Mixes of Chondrocytes and Human Dermal Fibroblasts

Primary human chondrocytes and human dermal fibroblasts were spun down and washed three times with DMEM+10% Human Serum. Cells were counted and resuspended in 1 ml differentiation medium (DMEM (highGlucose), Sodium Pyruvate, Proline 40 μg/ml, TGFβ5 ng/ml, Ascorbic acid 50 μg/ml, IGF1 10 ng/ml, ITS plus, HS 2%, Dexametazone 100 nM).

The cell suspension was diluted to prepare the cell pellets. Each cell pellet contained 5×10⁵ cells and a different percentage of fibroblasts (15%, 30%, 50% and 100%). The cell pellets were prepared in 15 ml conical test tubes. The test tubes were spun at 1000 rpm for 5 minutes to obtain cell pellets. The cell pellets were incubated at 37° C., 5% CO₂. Medium was changed three times a week.

Histological analysis: the cell pellets were analyzed three weeks after their preparation and stained with Alcian Blue (A stain designed to show Mucopolysaccharides or Glycosaminoglycans).

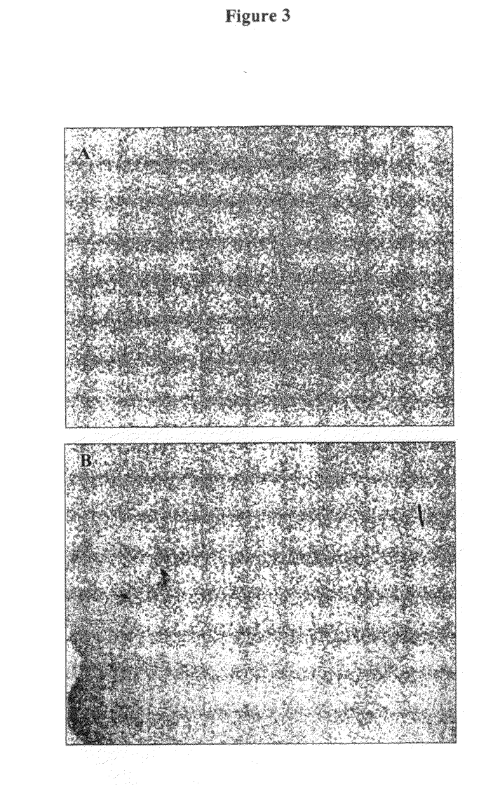

Cell pellets composed of 50% or less dermal fibroblasts had a similar solid consistency as those made of 100% chondrocytes. The control comprising 100% fibroblasts did not form a cell pellet culture. It shrank and became progressively smaller with time (FIG. 3).

### Example 5

### Mesenchymal Progenitor Cells

Mesenchymal progenitor cells (MPCs) are isolated and the populations enriched from bone marrow in a number of ways. In a non-limiting example, U.S. Pat. Nos. 6,645,727 and 6,517,872 teach methods for enriching MPCs. In general, mononuclear cells are separated by centrifugation in Ficoll-Hypaque gradients (Sigma; US), suspended in a-minimum essential medium (MEM) containing 20% FBS and seeded at a concentration of about 1×10⁶ cells/cm². After 3 days, nonadherent cells are removed by washing with phosphate-buffered saline (PBS), and the monolayer of adherent cells are cultured to confluency. The monolayer of cells is expanded by consecutive subcultivations in appropriate media at densities of about 5×10³ cells/cm².

A pellet of mesenchymal progenitor cells is prepared as described in example 3 above.

### Example 6

### Recombinant Viral Vectors

In certain embodiments cartilage explants and chondrocytes are transduced using viral vectors known in the art. In a non-limiting example, U.S. Pat. No. 6,803,234 discloses Adenovirus derivatives useful as gene delivery vectors for chondrocytes. In general, a nucleotide sequence encoding a therapeutic agent of choice, such as a peptide or protein, is cloned into a viral vector and the recombinant vector is used to transduce chondrocytes. In certain embodiments the therapeutic agent is an antibody. In other embodiments the therapeutic agent is a growth factor.

### Example 7

### Adenovirus Transduction of Human Primary Chondrocytes

Human primary chondrocytes are cultured in Dulbecco's modified Eagles medium (DMEM) supplemented with 10% fetal calf serum and further supplemented with essential amino acids (proline 0.4 mM), non-essential amino acids (1.times.), cholic acid-6-phosphate (0.2 mM) and buffered with HEPES (10 nM) (all materials derived from Gibco). In a first experiment, about 10⁵ chondrocytes are seeded in the wells of 24-well plates. The next day cells are exposed to either about 100, 500, or 1000 virus particles per cell of recombinant AV or AAV comprising a reporter gene, such as luciferase or lacZ. Forty-eight hours after the addition of virus, cells are washed twice with 1 ml PBS after which cells are lysed by adding 100 μl of cell lysis buffer. In cells transduced with a luciferase vector, luciferase activity is determined using a commercially available luciferase assay kit.

Cells that were infected with a recombinant vector comprising a lacZ reporter gene are used to determine the expression of the lacZ transgene over time. For this, cells are washed twice with PBS and fixed with 0.5 ml/well of a formaldehyde-gluteraldehyde fixative solution and incubated for 10 min. at room temperature. Cells are washed twice with PBS and stained with 0.5 ml/well staining solution (1 ml K₃Fe(CN)₆, 1 ml K₄Fe(CN)₆, 80 μl 1 M MgCl₂, brought to 40 ml with PBS. About 150 μl X-Gal per 6 ml is added prior to use) for 4 h at 37° C. Positive cells were counted and compared to negative cells.

### Example 8

### Expression of Green Fluorescent Protein in Human Chondrocytes Upon Infection by Adenovirus

Both luciferase and lacZ reporter proteins provide data concerning the transduction efficiency of AV and AAV in chondrocytes, but in certain instances it is important to determine the transduction at the level of individual cells. Therefore, chondrocytes are transduced with a vector carrying the green fluorescent protein (GFP) as a marker gene. Detection of GFP expression can be monitored using a flow cytometer. Vectors comprising GFP are used to transduce explants comprising chondrocytes and primary chondrocytes.

Human primary chondrocytes are seeded 24 h prior to infection in a density of about 10⁵ cells/well in a 24 well dish. Cells are exposed for 2 h to the Adenovirus vectors at a concentration of about 100, 500, 1000, virus particles per cell. Forty-eight hours after virus exposure cells are harvested and subjected to flow cytometric analysis. Non-transduced chondrocytes are used as a control (background gate 1% positive cells). Subsequently, cells exposed to the different recombinant vectors were assayed.

### Example 9

### A plasmid Vector Comprising Col 2 Promoter Directing GFP Expression

The Collagen 2A1 promoter was isolated from a construct with the NotI and ClaI restriction enzymes to obtain ˜6 kbp fragment. This fragment was cloned upstream to the GFP gene in the pEGFP-N1 (Clontech) vector. The pEGFP-N1 was digested with EcoRI-AseI to delete the CMV promoter and the Col 2A1 promoter was ligated at these sites. The resulted plasmid was named pEGFP-Col P.

In order to obtain a retroviral vector containing the Col 2A1-GFP fragment, the Col 2A1 promoter fragment (NotI-ClaI) was ligated into pLXSN (HpaI digested) in reverse orientation to the 5′LTR and in correct orientation to the 3′LTR (the 5′LTR can act as a promoter while the 3′LTR can't). The resulting vector was partially digested with EcoRI to get a linear plasmid for ligation of the GFP fragment. The GFP fragment was excised from pEGFP-N1 by digestion with AflII-EcoRI. The resulted plasmid was named pLXSN-Col P-GFP

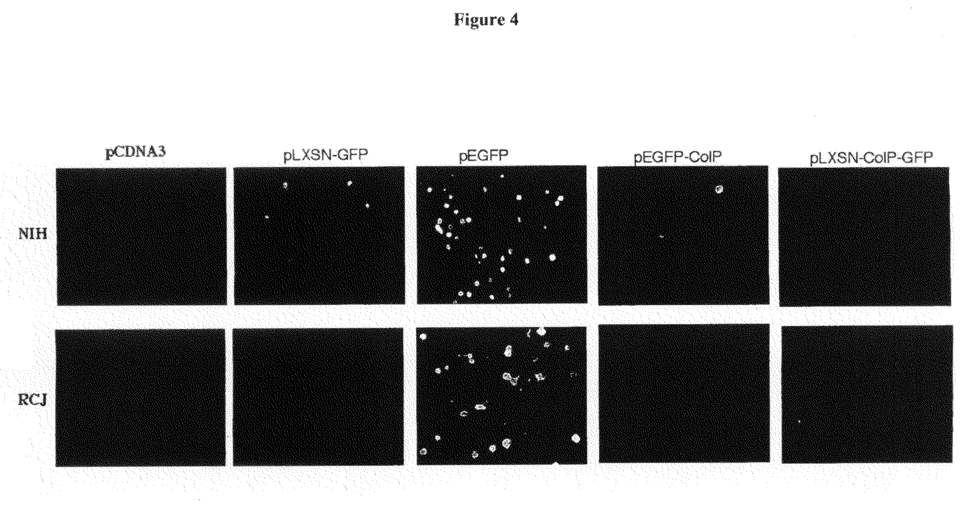

Transfection of chondrocytic cell lines, RCJ or RCS, was done using the Lipofectamine+ transfection reagent (GibcoBRL). Cells were seeded 24 h prior to transfection at 3-5×10⁵ cells/35 mm plate. A total of 2 μg DNA were mixed with the +reagent. The mixtures were incubated at room temperature for 15 min, the diluted lipofectamine was added and incubated for 30 min at room temperature and then added to the cells. After 3 h at 37° C., the transfection mixture was replaced with complete growth medium. Cells were harvested and assayed 48 h after transfection. GFP expression was visualized using an Olympus BX60 microscope. FIG. 4 shows the GFP expression in the transfected cells.

### Example 10

### In vitro Analysis of Gene Expression

Analysis of the gene product produced by the condensed chondrocyte mass is measured using laboratory techniques known in the art. Therapeutic proteins can be tested in ELISA assays, direct binding assays or functional assays.

### REFERENCES

Arai M, Anderson D, Kurdi Y, Annis-Freeman B, Shields K, Collins-Racie L A,
Corcoran C, DiBlasio-Smith E, Pittman D D, Domer A J, Morris E, LaVallie E R. Effect of adenovirus-mediated overexpression of bovine ADAMTS-4 and human ADAMTS-5 in primary bovine articular chondrocyte pellet culture system. Osteoarthritis Cartilage. (8):599-613, 2004
Arai Y, Kubo T, Fushiki S, Mazda O, Nakai H, Iwaki Y, Imanishi J, Hirasawa Y. Gene delivery to human chondrocytes by an adeno associated virus vector. J Rheumatol. 27(4):979-82, 2000
Coffin, J M, Hughes, S M, Varmus, H E, Eds., “Retroviruses” Cold Spring Harbor Laboratory Press: pp 758-763, 1997
Dinser R, Kreppel F, Zaucke F, Blank C, Paulsson M, Kochanek S, Maurer P. Comparison of long-term transgene expression after non-viral and adenoviral gene transfer into primary articular chondrocytes. Histochem Cell Biol. 116(1):69-77, 2001
Ikeda T, Kubo T, Nakanishi T, Arai Y, Kobayashi K, Mazda O, Ohashi S, Takahashi K, Imanishi J, Takigawa M, Hirasawa Y. Ex vivo gene delivery using an adenovirus vector in treatment for cartilage defects. J Rheumatol. (4):990-6 2000
Madry H, Cucchiarini M, Terwilliger E F, Trippel S B. Recombinant adeno-associated virus vectors efficiently and persistently transduce chondrocytes in normal and osteoarthritic human articular cartilage. Hum Gene Ther. 14(4):393-402 2003
Madry, H and Trippel, S B. Efficient lipid-mediated gene transfer to articular chondrocytes. Gene Ther. 7(4):286-91 2000
Pereboeva, L. Komarova, S. Mikheeva, G. Krasnykh, V. Curiel D T., Approaches to Utilize Mesenchymal Progenitor Cells as Cellular Vehicles. Stem Cells. 21:389-404, 2003
Ui-Tei, K., Naito, Y., Takahashi, F., Haraguchi, T., Ohni-Hamazaki, H., Juni, A., Ueda, R., and Saigo, K. (2004). Guidelines for the selection of highly effective siRNA sequences for mammalian and chick RNA interference. NAR 32(3):936-48.
Zhang Z, McCaffery J M, Spencer R G, Francomano C A. Hyaline cartilage engineered by chondrocytes in pellet culture: histological, immunohistochemical and ultrastructural analysis in comparison with cartilage explants. J. Anat. 205(3):229-37, 2004

It will be appreciated by a person skilled in the art that the present invention is not limited by what has been particularly shown and described hereinabove. Rather, the scope of the invention is defined by the claims that follow.

1. A cell mass comprising a plurality of genetically modified chondrocytes, wherein the genetically modified chondrocytes express a therapeutic agent.

2. The cell mass according to claim 1 selected from a mass formed from dispersed genetically modified chondrocytes, a genetically modified chondrocyte based explant, and a mass formed from cells derived from a genetically modified chondrocyte based explant.

3. The cell mass according to claim 1 wherein said chondrocytes are selected from primary chondrocytes and a chondrogenic cell line.

4. The cell mass according to claim 3 wherein said chondrocytes are derived from a source selected from articular cartilage, chondroprogenitor cells and mesenchymal progenitor cells (MPC).

5. The cell mass according to claim 1 wherein said chondrocytes are isolated from a source selected from an autologous source, an allogeneic source and a xenogeneic source.

6. The cell mass according to claim 1 further comprising non-chondrocytic cells wherein the cell mass substantially retains cartilage characteristics.

7. The cell mass according to claim 6 wherein the non-chondrocytic cells are selected from a group consisting of fibroblasts, endothelial cells, β islet cells, and liver cells.

8. (canceled)

9. The cell mass according to claim 1 wherein said chondrocytes are genetically modified using a gene delivery vehicle selected from a viral vector and a non-viral agent.

10. The cell mass according to claim 9 wherein the gene delivery vehicle is a viral vector selected from adenovirus, adeno-associated virus and a retrovirus.

11.-19. (canceled)

20. A method of transplanting to a subject in need of a therapeutic agent a cell mass comprising a plurality of genetically modified chondrocytes, the method comprising the steps of:
a. isolating a cartilage explant;
b. transducing cells of the explant to form genetically modified chondrocytes; and
c. transplanting the genetically modified chondrocytes into a subject, wherein the genetically modified chondrocytes express the therapeutic agent.

21. The method according to claim 20 wherein the cell mass is selected from a mass formed from dispersed genetically modified chondrocytes, a genetically modified chondrocyte based explant, and a mass formed from cells derived from a genetically modified chondrocyte based explant.

22. The method of claim 20 further comprising the step of dispersing chondrocytes from the explant prior to transducing them.

23. The method of claim 20 further comprising condensing the cell mass prior to transplanting it into a subject.

24. The method according to claim 20 wherein said chondrocytes are selected from primary chondrocytes and a chondrogenic cell line.

25. The method according to claim 20 wherein said chondrocytes are isolated from a source selected from an allogeneic source, an autologous source and a xenogeneic source.

26. The method according to claim 20 wherein said cell mass further comprises non-chondrocytic cells and wherein the cell mass substantially retains cartilage characteristics.

27. The method according to claim 26 wherein the non-chondrocytic cells are selected from a group consisting of fibroblasts, endothelial cells, P islet cells, and liver cells.

28. (canceled)

29. The method according to claim 20 wherein said chondrocytes are genetically modified using a gene delivery vehicle selected from a viral vector and a non-viral agent.

30. The method according to claim 26 wherein the gene delivery vehicle is a viral vector selected from adenovirus, adeno-associated virus and a retrovirus.

31. The method cell mass according to claim 20 wherein the therapeutic agent is useful for treating a disease or disorder in a subject.

32. The method according to claim 31 wherein the therapeutic agent is selected to induce or stimulate a cellular function selected from cell division, cell growth, cell proliferation and cell differentiation.

33. The method according to claim 31 wherein the therapeutic agent is selected to inhibit a cellular function selected from cell division, cell growth, cell proliferation and cell differentiation.

34. The method according to claim 31 wherein the therapeutic agent is selected from a peptide, a protein and a RNAi.

35. The method according to claim 34 wherein the therapeutic peptide or protein is selected from a growth factor, a growth factor receptors, a hormone, an antibody, a ribozyme, a protein hormone, a peptide hormone, a cytokine, a cytokine receptor, a pituitary hormone, a clotting factor, an anti-clotting factor, a plasminogen activator, an enzyme, an enzyme inhibitor, an extracellular matrix protein, an immunotoxin, a surface membrane protein, a T-cell receptor transport protein, a regulatory proteins and fragments thereof.

36. The method according to claim 35 wherein the therapeutic protein is antibody.

37. The method according to claim 31 wherein the disease or disorder is an acquired or genetic deficiency.

38. The method according to claim 31 wherein the disease or disorder is an acquired or genetic gain of function disease or disorder.

39. The method according to claim 31 wherein the disease or disorder is selected from a cartilage or bone disease or disorder, a brain disorder, a cardio-vascular disorder, a pulmonary disorder, a muscular disorder, a lymphatic system disorder.

40. A method of transplanting to a subject in need of a therapeutic agent an implant comprising genetically modified chondrocytes, wherein the genetically modified chondrocytes express the therapeutic agent, wherein said chondrocytes are derived from a source selected from articular cartilage, chondroprogenitor cells and mesenchymal progenitor cells (MPC).
